(19) United States
(12) Patent Application Publication
(10) Pub. No.: US 2006/0094984 A1
(43) Pub. Date: May 4, 2006

(54) Urine cell sample enhancement

(76) Inventors: Nathan P. Wood, Winchendon, MA (US); Robert Sakal, Bolton, MA (US)

Correspondence Address: CYTYC CORPORATION, 250 CAMPUS DRIVE, MARLBOROUGH, MA, 01752, US

(21) Appl. No.: 10/975,160

(22) Filed: Oct. 28, 2004

(51) Int. Cl. A61B 5/00; A61N 1/08

(52) U.S. Cl. 600/573; 607/40

(57) ABSTRACT

The present invention relates to a method of increasing the yield of cells collected in a urine sample comprising the steps of applying energy wave forms to a bladder wherein the energy loosens cells attached to the inner lining of the bladder, and collecting urine sample containing the loosened cells.

### FIELD OF THE INVENTION

The present invention relates to methods for the increasing the yield of cells collected in a urine sample. In particular, the method of the present invention increases the number of cells obtained from urine or bladder washings by the use of energy wave forms which loosen cells from the inner lining of the bladder.

### A BACKGROUND OF THE INVENTION

Cancer of the bladder is the fifth most common cancer in the United States with an annual incidence of about 18 cases per 100,000 or over 50,000 new cases per year, leading to more than 10,000 deaths annually. The incidence (80% of the cases) is highest in the 50-79 year age group; the disease prevalence peaks in the seventh decade of life with a strong male predominance. Bladder cancer accounts for 7% of all new cases of cancer among men and 3% among women, as well as 2% of cancer deaths among men and 1% among women. Occupational exposure may account for 21-25% of bladder cancer in white males in the United States. Males are affected three to four times more frequently than females and over half of all deaths from bladder cancer occur after age 70. There were 12,710 estimated deaths for urinary bladder cancer in the US in 2004 (Cancer Facts and Figures, American Cancer Society, 2004).

Survival in patients with bladder cancer is strongly associated with stage at diagnosis. Although most cancers are superficial at time of diagnosis, currently 10-20% of all cases of bladder cancer have invaded the muscular wall of the bladder when first diagnosed, with a much worse prognosis. Five-year survival for patients with superficial disease is over 90%, but falls to less than 50% with invasive disease (Wingo P A, Tong T, Bolden S. Cancer Statistics, Calif. Cancer J Clin 1995;45:8-30). The rationale for screening is that detecting and treating early asymptomatic bladder cancers may prevent progression to invasive disease, or allow for more effective treatment of noninvasive tumors, which have a high rate of recurrence. Many cases detected on screening, however, are low-grade transitional cell cancers with low propensity for invasion; in contrast, since aggressive cancers may invade early, periodic screening may have a limited potential for detecting lethal bladder cancers at an early, treatable stage

Thus, it is not only important to detect the presence of tumor early, it is also crucial to identify the high-grade tumors which present with a grim prognosis. Tumor recurrence is also a characteristic of bladder carcinoma. Therefore, despite a complete remission of the original tumor, patients must be closely followed in order to monitor the treatment efficacy and recurrence (Heney, Natural history of bladder cancer. Urol. Clin. North Am., 19: 429-433, 1992).

The current methods for bladder cancer detection involve cystoscopy, bladder washings, and biopsy. These procedures are invasive and require some form of anesthesia. Urine cytology use is possible but its specificity is low due to the low number of cells contained in a urine sample.

The present invention relates to a method of increasing the yield of cells collected in a urine sample. In particular, the method of the present invention increase in cells obtained from urine or bladder washings by use of energy wave forms which help loosen cells from the inner lining of the bladder.

### SUMMARY OF THE INVENTION

This invention generally relates to a method of increasing the yield of cells collected in a urine sample, said method comprises the steps of applying energy wave forms to a bladder wherein said energy loosens cells attached to the inner lining of the bladder, and collecting urine sample containing said loosened cells.

In another aspect of the present invention, said energy wave forms are vibration, sound, ultrasound, or heat waves or a combination thereof.

### DETAILED DESCRIPTION OF THE INVENTION

Almost all cases of bladder cancer (>90%) are transitional cell carcinomas (TCCs), which are malignant, usually papillary, tumors derived from transitional stratified epithelium. For example, some TCCs behave in a benign fashion (low-grade, G1 tumors) whereas others are intermediate (G2 tumors) to highly aggressive (G3 tumors and carcinoma in situ (CIS)). The high-grade tumors generally metastasize quickly; indeed, at the time of clinical presentation (e.g., hematuria, irritative voiding symptoms etc.), invasive disease already exists for many patients with high-grade bladder tumors.

Of special interest is carcinoma-in-situ (CIS) of the bladder, a lesion presenting problems in diagnosis and of unpredictable behavior (e.g. recurrence and progression) and where morphologic definition is arbitrary and generally defined as a total replacement of the urothelial surface by cells which bear morphologic features of the carcinoma, but which lack architectural alterations other than an increase in the number of cell layers, i.e., a flat lesion.

The current methods for bladder cancer detection involve cystoscopic biopsy and urine cytology. Early asymptomatic bladder cancer may be associated with occult bleeding (microscopic hematuria) or the presence of dysplastic cells in the urine. The definition of significant hematuria varies, but more than 3-5 red blood cells (RBCs) per high-powered field in microscopic analysis of the urine sediment is usually considered abnormal. Cystoscopy along with cytology is the most commonly accepted method for diagnosing bladder cancer. In order to diagnose transitional cell carcinoma in the bladder, it is necessary to do a cystoscopic biopsy. A biopsy is the removal of a small sample of living tissue from an organ, such as the bladder, for microscopic examination to confirm or establish a diagnosis, estimate prognosis, or follow the course of a disease. Biopsies are invasive procedures, and are therefore not desirable as it is necessary for a person undergoing biopsy to undergo anesthesia and can be uncomfortable and may require individuals to be admitted into hospitals. In addition, as with any invasive procedure, an individual undergoing a biopsy runs the risk of infection. Further, the entire bladder cannot be biopsied to determine whether bladder cancer is present.

Once obtained, a biopsy can be analyzed histologically and for DNA content. However, that approach is not always successful, particularly when a limited number of cells are present in the sample. In particular, numerical chromosomal structural changes have been often reported. Unfortunately, cytogenetic results on bladder cancer are often difficult to obtain due to the poor growth of tumor cells in culture. In these cases, fluorescence in situ hybridization (FISH) has been used as an alternative technique in detecting numerical chromosome changes. To date, FISH has been performed on cultured cells and paraffin embedded tissue sections of bladder tumors with good results.

Cystoscopy is currently considered the “gold standard” for identification of bladder tumors. Although it is effective in identifying visible tumors in the bladder, cystoscopy is invasive. For this reason, urine cytology is often used to detect tumors, because it is a simpler and noninvasive alternative. Urine cytology microscopically identifies the presence of abnormal cells which are shed into the urine in patients with bladder cancer. The method has high specificity (i.e., few false-positives), however, it has low sensitivity (i.e., many false-negatives, especially in superficial and low-grade tumors) (see Pode, D., et al., J Urol 1998;159(2):389-93; Landman, J., et al. Urology 1998;52(3):398-402) and results are not immediately available and are interpreter-dependent.

One method for decreasing the false negative rate of urine cytology would be to increase the number of cells obtained in a urine sample and thus increase the chances of finding cancerous cells. The method of the present invention describes new methods of increasing the number of cells obtained from urine or from bladder washings by the application of energy wave forms to the bladder which in turn facilitates the exfoliation of cells from the inner lining of the bladder.

Waves come in many shapes and forms. One way to categorize waves is on the basis of the direction of movement of the individual particles of the medium relative to the direction that the waves travel. A wave is an energy transport phenomenon that transports energy along a medium without transporting matter.

A transverse wave is a wave in which particles of the medium move in a direction perpendicular to the direction that the wave moves.

A longitudinal wave is a wave in which particles of the medium move in a direction parallel to the direction that the wave moves. A sound wave is a classic example of a longitudinal wave.

A sound wave is an example of a mechanical wave. Mechanical waves require a medium in order to transport their energy from one location to another. A sound wave is the pattern of disturbance caused by the movement of energy traveling through a medium (such as air, water, or any other liquid or solid matter) as it propagates away from the source of the sound. The source is some object that causes a vibration, such as a ringing telephone, or a person's vocal chords. The vibration disturbs the particles in the surrounding medium; those particles disturb those next to them, and so on. The pattern of the disturbance creates outward movement in a wave pattern, like waves of seawater on the ocean. The wave carries the sound energy through the medium, usually in all directions and less intensely as it moves farther from the source.

The frequency of a wave refers to how often the particles of the medium vibrate when a wave passes through the medium.

The period of a wave is the time for a particle on a medium to make one complete vibrational cycle. Period, being a time, is measured in units of time such as seconds, hours, days or years.

The amount of energy carried by a wave is related to the amplitude of the wave. A high-energy wave is characterized by high amplitude; a low energy wave is characterized by low amplitude. In the case of a wave, the speed is the distance traveled by a given point on the wave (such as a crest) in a given interval of time.

Sound wave forms could be used at low frequency to create vibrations capable of traveling longitudinally through human tissues to the bladder that would help facilitate the exfoliation of cells from the inner lining of the bladder.

A varied range of ultrasonic frequencies may also agitate the bladder lining, helping to exfoliate cells. The vibration of waveforms reaching the inner lining of the bladder is the principal method of helping to loosen bladder cells as mentioned above.

Another wave form that may be useful in the method of the present invention is therapeutic acoustic energy. Acoustic energy may be applied at a specific range of frequencies to deliver vibrational characteristics to help with cell exfoliation from the lining of the bladder.

Another approach to loosen cells from the lining of the bladder would be to use ExMI (Extracorporeal Magnetic Innervation), which induces nerve impulses that, when applied to the pelvic floor, will exercise muscles that control bladder functions. This technique could be used for the purpose of shaking loose (shedding) additional cells from the inner lining of the bladder via the muscle contractions that control the bladder.

Other such non-mechanical methods include specific exercises (leg raises etc.) or massage techniques could in turn help exfoliate the cells from the inner lining of the bladder. The addition of a heating source may also assist in exfoliation of the bladder cells.

Once the bladder cells have been isolated, the cells may be examined by molecular various ancillary modalities for detection of malignancies such as Flow cytometry, bladder tumor antigen (BTA), nuclear matrix protein (NMP), matrix metalloproteinase (MMP), human chorionic gonadotrophic (HCG), telomerase, and other techniques

A large number of potential molecular markers of bladder cancer have been identified, although only a few are truly independent prognostic factors. A number of markers may need to be measured in a single tumor and used as a combination for use in the diagnosis and prognosis of transitional cell carcinoma (TCC). Several urinary markers and tests such as BTA Stat, BTA TRAK, NMP22, telomerase, HA and HAse tests, Immunocyt, Quanticyt, FDP, BLCA-4, FISH, CYFRA-21-1. Epidermal growth factor receptor immunoreactivity has been shown to be an independent predictor of survival and stage progression. TP53 may be an independent predictor of recurrence and overall survival in TCC confined to the bladder, and TP53 alterations may predict chemosensitivity in patients who have had TCC treated by radical cystectomy.

Various methods for analyzing exfoliated cells have been developed to detect genetic alterations, tumor suppressor genes, oncogenes, tumor cell products, and angiogenic factors. It is known that cancer progression in stage or grade is associated with increasing chromosomal anomalies that can be assessed by measuring tumor cell DNA content, by cytogenetic studies, or by measuring the function of activation in oncogenes and inactivation of tumor suppressor genes. For instance, Masters et al., (“DNA Ploidy and the Prognosis of Stage pT1 Bladder Cancer,” Br. J. Urolo, 64, 403 (1985)), used DNA measurements to show a correlation to tumor grade and recurrence rates. Norming et al., (“Deoxyribonucleic Acid Profile and Tumor Progression in Primary Carcinoma in situ of the Bladder: A Study of 63 Patients with Grade 3 Lesions,” J. Urol. 147, 11 (1992)), suggests that tile number of aneuploid cell populations is an indicator for tumor progression.

Cytogenetic analyses of bladder cancer have revealed recurrent abnormalities affecting several chromosomes, particularly structural rearrangements of chromosomes 5 and 9 and numerical changes of chromosome 7, 8, 9 and Y. Rearrangements of chromosomes 1, 10 and 11 have also been reported.

A few other markers such as DNA ploidy, p53 mutations, microsatellite DNA, beta.-glucuronidase, basic-FGF levels, autocrine motility factor receptor etc. have been shown to be associated with bladder cancer (Sidransky and Messing, Molecular genetics and biochemical mechanisms in bladder cancer. Urol. Clin. North Am., 19: 629-639, 1992; Mao et al., Molecular detection of primary bladder cancer by microsatellite DNA. Science, 271: 659-662, 1996; Nguyen et al., Elevated levels of the angiogenic peptide basic fibroblast growth factor in urine of bladder cancer patients. J. Natl. Cancer Inst., 85: 241-242, 1993; Esrig et al., Accumulation of nuclear p53 and tumor progression in bladder cancer. N. Engl. J. Med., 331: 1259-1264, 1994; Ho, Urinary beta.-glucuronidase in screening and follow up of primary urinary tract malignancy. J. Urol., 154: 1335-1338, 1995; Korman et al., Autocrine motility factor receptor as a possible urine marker for transitional cell carcinoma of the bladder. J. Urol., 154: 347-349, 1995). However, most of these have not yet been used clinically as diagnostic markers.

Recently, FISH has become the best alternative method to cytogenetic analysis of bladder cancer. Various studies have described numerical changes of chromosomes 7(+7), 8(+8), 9(−9) and Y(−Y) and less frequently 10 and 11.

The cells to be analyzed according to the present method are obtained from either the urine or the bladder washings from a patient whose cells are desired to be analyzed. The cells are then subjected to in situ hybridization with nucleic acid probes suitable for the analysis in question. In a preferred embodiment of the present invention, the cells are analyzed to detect the presence of bladder cancer or carcinoma-in-situ. In each case, cells are obtained from a patient suspected of having, or to be tested for, those diseases. If the cells are to be obtained from urine, it is preferable to obtain them from the patient's first morning urine. A sufficient amount should be collected in order to obtain a suitable number of cells for analysis. A suitable amount is in the range of about 50 to about 100 ml of urine. If the cells are obtained from bladder washings, a suitable amount of washings to be collected is also in the range of about 50 to about 100 ml. The washing medium may be any liquid conventionally used, such as water and preferably saline solution.

The cells contained in the urine or bladder washings may be harvested in any suitable way, including commercially available urine collection kits such as the ThinPrep® UroCyte™ Urine Collection Kit (Cytyc Corporation, Boxborough, Mass.). Once the cells are harvested, they may be prepared for in situ hybridization by methods well known to one of ordinary skill. The cells may be analyzed within a short time after harvesting, or they may be fixed and stored for a longer period of time before analysis. The cells may be fixed by any suitable known fixative, such as CytoLyt™ (Cytyc Corporation, Boxborough, Mass.). For in situ hybridization analysis, the cells are placed on a solid support suitable for examination by microscopy, such as a slide or coverslip, and treated by methods well known in the art to permeablize the cells so that detectable probe can enter the cells and bind to the chromosomal region.

Examination of the cellular content of the urine collected using the method of the present invention may also be used to diagnose other diseases. For example, a screening test for kidney failure can be conducted based upon the presence of erythrocytes, leukocytes, epithelial cells, casts and bacteria in urine. Measurement of erythrocytes is important in terms of determining whether hemorrhage has occurred in the tract from the slomerulus to the urethra of the kidney. The appearance of leukocytes is considered to be a possible indication of a kidney disorder such as pyelonephritis, and detection thereof is important in early discovery of inflammation and infection. Furthermore, by examining cast and erythrocyte morphology, the origin of such inflammation and infection, namely the abnormal parts of the body, can be surmised.

The in situ hybridization of the present invention may be carried out in ways well known to the person skilled in the art. For example, a hybridization solution comprising at least one detectable nucleic acid probe capable of hybridizing to a chromosome within the cell is contacted with the cell under hybridization conditions. Any hybridization is then detected, and then compared to a predetermined hybridization pattern from normal or control cells. It is preferred to use a nucleic acid probe which will selectively hybridize to only one chromosome. By selectively hybridize is meant that the probe will bind to a particular chromosome in an amount sufficient to detect the chromosome, without binding sufficiently to other chromosomes to allow identification of such other chromosomes. It is preferred that a probe be used that selectively binds to a chromosome that undergoes a numerical change in bladder cancer or carcinoma in situ. For example, one or more probes to chromosomes 1, 7, 8, 9, 10, 11, 17, Y and X may be used. Preferably, the probes are alpha-centromeric probes for the chromosomes listed above. Those probes are readily commercially available (Oncor, Inc., Gaithersburg, Md.). In a preferred embodiment, the hybridization solution contains a multiplicity of probes, each specific for a different chromosome. For example, the hybridization solution may contain an amount of a chromosome 7 probe and an amount of a chromosome 8 probe. Other possible combinations are apparent and within the scope of the present invention.

The probes may be prepared by any method known in the art, including synthetically or grown in a biological host. Synthetic methods include oligonucleotide synthesis, riboprobes, and PCR.

The probe may be labeled with a detectable marker by any method known in the art. Methods for labeling probes include random priming, end labeling, PCR and nick translation. Enzymatic labeling is conducted in the presence of nucleic acid polymerase, three unlabeled nucleotides, and a fourth nucleotide which is either directly labeled, contains a linker arm for attaching a label, or is attached to a hapten or other molecule to which a labeled binding molecule may bind. Suitable direct labels include radioactive labels such as .sup.32 P, .sup.3H, and .sup.35 S and non-radioactive labels such as fluorescent markers, such as fluorescein, Texas Red, AMCA blue, lucifer yellow, rhodamine, and the like; cyanin dyes which are detectable with visible light; enzymes and the like. Labels may also be incorporated chemically into DNA probes by bisulfite-mediated transamination or directly during oligonucleotide synthesis.

Specifically, fluorescent markers may be attached to nucleotides with activated linker arms which have been incorporated into the probe. Probes may be indirectly labeled by the methods disclosed above, by incorporating a nucleotide covalently linked to a hapten or other molecule such as biotin or digoxygenin, and performing a sandwich hybridization with a labeled antibody directed to that hapten or other molecule, or in the case of biotin, with avidin conjugated to a detectable label. Antibodies and avidin may be conjugated with a fluorescent marker, or with an enzymatic marker such as alkaline phosphatase or horseradish peroxidase to render them detectable. Conjugated avidin and antibodies are commercially available from companies such as Vector Laboratories (Burlingame, Calif.) and Boehringer Mannheim (Indianapolis, Ind.).

The enzyme can be detected through a calorimetric reaction by providing a substrate for the enzyme. In the presence of various substrates, different colors are produced by the reaction, and these colors can be visualized to separately detect multiple probes. Any substrate known in the art may be used. Preferred substrates for alkaline phosphatase include 5-bromo-4-chloro-3-indolylphosphate (BCIP) and nitro blue tetrazolium (NBT). The preferred substrate for horseradish peroxidase is diaminobenzoate (DAB).

Fluorescently labeled probes suitable for use in the in situ hybridization methods of the present invention are preferably in the range of 150-500 nucleotides long. Probes may be DNA or RNA, preferably DNA.

Hybridization of the detectable probes to the cells is conducted with a probe concentration of 0.1-500 ng/μl, preferably 5-250 ng/μl. The probe concentration is greater for a larger clone. The hybridization mixture will preferably contain a denaturing agent such as formamide. In general, hybridization is carried out at 25-45° C., more preferably at 32-40° C., and most preferably at 37-38° C. The time required for hybridization is about 0.25-96 hours, more preferably 1-72 hours, and most preferably for 4-24 hours. Hybridization time will be varied based on probe concentration and hybridization solution content which may contain accelerators such as hnRNP binding protein, trialkyl ammonium salts, lactams, and the like. Slides are then washed with solutions containing a denaturing agent, such as formamide, and decreasing concentrations of sodium chloride or in any solution that removes unbound and mismatched probe.

The temperature and concentration of salt will vary depending on the stringency of hybridization which is desired. For example, high stringency washes may be carried out at 42-68° C., while intermediate stringency may be in the range of 37-55° C., and low stringency may be in the range of 30-37° C. Salt concentration for a high stringency wash may be 0.5-1.0×SSC (0.15M NaCl, 0.015M Na citrate), while medium stringency may be 1× to 4×, and low stringency may be 2× to 6×SSC.

The detection incubation steps, if required, should preferably be carried out in a moist chamber at 23-42° C., more preferably at 25-38° C. and most preferably at 37-38° C. Labeled reagents should preferably be diluted in a solution containing a blocking reagent, such as bovine serum albumin, non-fat dry milk, or the like. Dilutions may range from 1:10-1:10,000, more preferably 1:50-1:5,000, and most preferably at 1:100-1:1,000. The slides or other solid support should be washed between each incubation step to remove excess reagent.

Slides may then be mounted and analyzed by microscopy in the case of a visible detectable marker, or by exposure to autoradiographic film in the case of a radioactive marker. In the case of a fluorescent marker, slides are preferably mounted in a solution which contains an antifade reagent, and analyzed using a fluorescence microscope. Multiple nuclei may be examined for increased accuracy of detection.

The invention can be embodied in other specific forms without departing from the spirit or essential characteristics thereof. The present embodiments are therefore to be considered in all respects as illustrative and not restrictive. The scope of the invention is indicated by the appended claims, rather than by the foregoing description, and all changes which come within the meaning and range of equivalency of the claims are therefore intended to be embraced therein.

1. A method of increasing the yield of cells collected in a urine sample, said method comprising the steps of applying energy wave forms to a bladder wherein said energy loosens cells attached to the inner lining of the bladder, and collecting urine sample containing the loosened cells.

2. The method of claim 1 wherein said energy wave forms are vibration, sound, ultrasound, or heat waves or a combination thereof.

3. The method of claim 1 wherein the energy wave form is applied from internally

4. The method of claim 1 wherein the energy wave form is applied externally.
